(19) United States
(12) Patent Application Publication
(10) Pub. No.: US 2012/0165687 A1
(43) Pub. Date: Jun. 28, 2012

(54) SPHYGMOMANOMETER

(75) Inventors: Yukiya Sawanoi, Nara-shi (JP); Shingo Yamashita, Kyoto-shi (JP); Toshiaki Yuasa, Moriyama-shi (JP)

(73) Assignee: OMRON HEALTHCARE CO., LTD., Kyoto (JP)

(21) Appl. No.: 13/338,551

(22) Filed: Dec. 28, 2011

(30) Foreign Application Priority Data
Dec. 28, 2010 (JP) 2010-292742

(51) Int. Cl. A61B 5/022

(52) U.S. Cl. 600/499

(57) ABSTRACT

A blood pressure measurement device includes a sensor that detects a change of an internal pressure of an air bladder during the inflation and/or deflation of the air bladder. The sensor further includes a first sensor and a second sensor, which each have a diaphragm connected to the air bladder. A face of each of the diaphragms of the first sensor and the second sensor is flexibly displaced in accordance with the change of the internal pressure of the air bladder. The face of the diaphragm of the second sensor is arranged in a different position and/or direction from the face of the diaphragm of the first sensor. A central processing unit of the device includes a failure judgment unit that determines whether there was any failure with the sensor based on difference between the internal pressures detected by the first sensor and the second sensor.

### BACKGROUND OF INVENTION

The present invention relates to a blood pressure measurement device, particularly a blood pressure measurement device in which an air bladder is wrapped around and compresses the measurement site when blood pressure is measured.

Since the past, blood pressure measurement devices have been equipped with a sensor for measuring blood pressure, and blood pressure has been measured based on the output of the sensor. To accurately measure blood pressure using such a blood pressure measurement device, it is necessary to reliably detect when a failure occurs in the sensor.

For this purpose, Patent Reference 1 (JP-A-02-19133) discloses a technique for detecting a pressure sensor failure in a blood pressure measurement device that uses a pressure sensor, whereby a plurality of pressure sensors are installed and the detected values of the sensors are compared. The disclosure of the patent reference is incorporated herein by reference.

However, it is assumed that if failures occur in both of two pressure sensors, it is not possible to detect that failures have occurred in both pressure sensors even if the detected values of the pressure sensors are compared according to the art described in Patent Reference 1. Specifically, for example, if it is judged that no failures have occurred in both of two pressure sensors, as long as the pressure values measured by the two pressure sensors are equal or the difference between them does not exceed a prescribed value, their pressure values may be equal or the difference between them may not exceed the prescribed value if the same failure has occurred in both pressure sensors. In Patent Reference 1, the worst case due to a failure occurring in the pressure sensor is prevented by referencing another value such as power supply voltage.

The present invention was conceived while considering the above facts, and its objective is to reliably detect when a failure occurs in a sensor for measuring blood pressure in a blood pressure measurement device.

### SUMMARY OF INVENTION

According to one or more embodiments of the invention, a blood pressure measurement device includes a cuff containing an air bladder for wrapping around the measurement area of a subject; an air charger that inflates the air bladder; an air discharger that deflates the air bladder; and a sensor that detects a change of an internal pressure of the air bladder during the inflation and/or deflation of the air bladder. The sensor further includes a first sensor having a diaphragm connected to the air bladder, a face of the diaphragm is flexibly displaced in accordance with the changes of the internal pressure of the air bladder; and a second sensor having a diaphragm connected to the air bladder, a face of the diaphragm is flexibly displaced in accordance with the change of the internal pressure of the air bladder. The face of the diaphragm of the second sensor is arranged in a different position and/or direction from the face of the diaphragm of the first sensor. The blood pressure measurement device further includes a main body having a central processing unit that calculates the blood pressure of a measurement subject from the change of the internal pressure of the air bladder detected by the sensor, the central processing unit further comprising a failure judgment unit that determines whether there was any failure with the sensor. Upon receiving the internal pressure of the air bladder detected by the first sensor and the second sensor, the failure judgment unit determines whether a difference between the internal pressures detected by the first sensor and the second sensor is within a predetermined range. If the difference is beyond the predetermined range, the failure judgment unit determines that the sensor failed to perform normal detection of the internal pressure of the air bladder.

According to one or more embodiments of the invention, a blood pressure measurement device includes means for wrapping the air bladder around the measurement area of a subject; means for inflating the air bladder; means for deflating the air bladder; sensor means for detecting a change of an internal pressure of the air bladder during the inflation and/or deflation of the air bladder. The sensor means further includes a first sensor having a diaphragm connected to the air bladder, a face of the diaphragm is flexibly displaced in accordance with the changes of the internal pressure of the air bladder; and a second sensor having a diaphragm connected to the air bladder. A face of the diaphragm is flexibly displaced in accordance with the change of the internal pressure of the air bladder, the face of the diaphragm of the second sensor is arranged in a different position and/or direction from the face of the diaphragm of the first sensor; and means for calculating the blood pressure of a measurement subject from the change of the internal pressure of the air bladder detected by the sensor, the means further comprising means for determining whether there was any failure with the sensor. Upon receiving the internal pressure of the air bladder detected by the first sensor and the second sensor, a difference between the internal pressures detected by the first sensor and the second sensor is determined whether it is within a predetermined range. If the difference is beyond the predetermined range, the sensor is found to have failed to perform normal detection of the internal pressure of the air bladder.

According to one or more embodiments of the invention a method of detecting a failure of a sensor of blood pressure measurement device includes: wrapping an air bladder around the measurement area of a subject; inflating and/or deflating the air bladder; and detecting a change of an internal pressure of the air bladder during the inflation and/or deflation of the air bladder by using a sensor. The sensor includes: a first sensor having a diaphragm connected to the air bladder, a face of the diaphragm is flexibly displaced in accordance with the changes of the internal pressure of the air bladder; and a second sensor having a diaphragm connected to the air bladder, a face of the diaphragm is flexibly displaced in accordance with the change of the internal pressure of the air bladder, the face of the diaphragm of the second sensor is arranged in a different position and/or direction from the face of the diaphragm of the first sensor. Upon receiving a change of internal pressure of the air bladder, whether a difference between the internal pressures detected by the first sensor and the second sensor is within a predetermined range is determined. If the difference is beyond the predetermined range, determining that the sensor has failed to perform normal detection of the internal pressure of the air bladder.

According to one or more embodiments of the invention, because a plurality of pressure sensors are arranged such that the weight of the sensors onto the diaphragms of respective pressure sensors become different from each other, displacement of the diaphragms in response to the change of internal air pressure of the air bladder would be different from each other. As a result, output of these pressure sensors would be different from each other. That difference can be used to determine whether the sensor is working normally without failure. Because of the different condition/output of the plurality of pressure sensors, even if all of the sensors failed, the resulting output from those sensors are less likely to become the same. Therefore, the device can detect the failure of the pressure sensor more reliably than the blood pressure measurement device having pressure sensors that are arranged in a same condition or state.

### BRIEF DESCRIPTION OF DRAWINGS

FIG. 1 is a perspective view illustrating a specific example of the exterior of a blood pressure measurement device (sphygmomanometer) according to an embodiment of the present invention.

FIG. 2 is a schematic cross-sectional view of the sphygmomanometer of FIG. 1 during blood pressure measurement.

FIG. 3 is a block diagram of the sphygmomanometer of FIG. 1.

FIG. 4 is a table showing sample measurement record data pertaining to a user's blood, stored in the memory of FIG. 3.

FIGS. 5A and 5B are perspective views of the first pressure sensor of FIG. 3.

FIG. 6 is an exploded perspective view of the first pressure sensor of FIGS. 5A and 5B.

FIG. 7 is a schematic cross-sectional view of the first pressure sensor of FIG. 6.

FIG. 8 is a drawing for describing the arrangement of the first pressure sensor and the second pressure sensor in the sphygmomanometer of FIG. 1.

FIG. 9 is a drawing for describing the arrangement of the first pressure sensor and the second pressure sensor in the sphygmomanometer of FIG. 1.

FIG. 10 is a graph showing an example of the changes in capacitance of the first pressure sensor and the second pressure sensor when the pressure in the air bladder changes.

FIG. 11 is a flow chart of the blood pressure measurement process executed in the sphygmomanometer of FIG. 1.

FIG. 12 is a flow chart of a pressure sensor status detection process subroutine of FIG. 11.

FIGS. 13A, 13B, and 13C are drawings illustrating sample displays of the display unit of the sphygmomanometer in embodiment (2) of the sphygmomanometer of FIG. 11.

FIG. 14 is a drawing for describing the attachment configuration of the plurality of pressure sensors in embodiment (2) of the sphygmomanometer of FIG. 1.

FIG. 15 is a drawing for describing the attachment configuration of the plurality of pressure sensors in embodiment (2) of the sphygmomanometer of FIG. 1.

FIG. 16 is a drawing illustrating the exterior of the blood pressure measurement device in embodiment (3) of the sphygmomanometer of FIG. 1.

FIG. 17 is a drawing for describing the configuration of the pressure sensor in embodiment (4) of the sphygmomanometer of FIG. 1.

FIG. 18 is a drawing for describing the attachment configuration of the plurality of pressure sensors in embodiment (5) of the sphygmomanometer of FIG. 1.

### DETAILED DESCRIPTION OF INVENTION

Embodiments of the present invention will be described below while referring to the drawings. In the descriptions below, the same parts and same constituent elements are given the same reference numerals. Their names and functions are also the same.

### First Embodiment

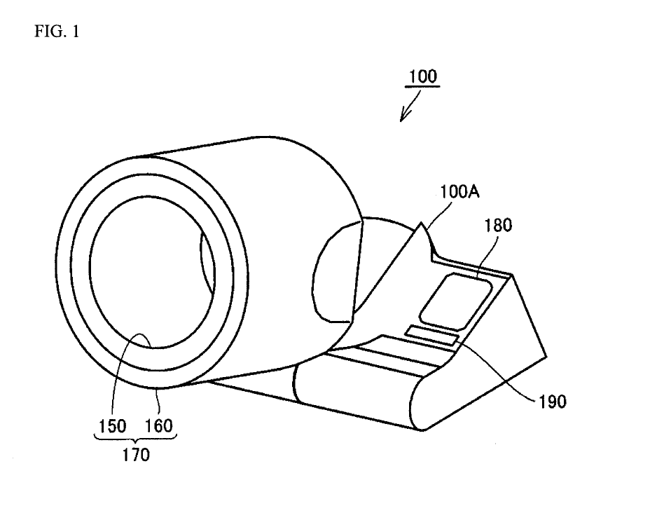

FIG. 1 is a perspective view illustrating a specific example of the exterior of a blood pressure measurement device (called “sphygmomanometer” hereinafter) 100 according to an embodiment of the present invention.

Referring to FIG. 1, the sphygmomanometer 100 according to this embodiment primarily comprises a main body 100A mounted on a table or the like, and a measurement unit 170 for insertion of the upper arm, which is the measurement site of the measurement subject. The upper part of the main body 100A is equipped with an operating unit 190 on which the power switch, measurement switch and so forth are arranged, a display unit 180, and an elbow rest. Furthermore, the measurement unit 170 is attached such that its angle with respect to the main body 100A can be varied, and is equipped with a housing 160, which is a substantially cylindrical casing, and a body compression fixing device housed inside the housing 160.

As shown in FIG. 1, in the normal state of use, the body compression fixing device housed inside the housing 160 is covered by a cover without being exposed. The display unit 180 is a known display unit such as a liquid crystal display, for example.

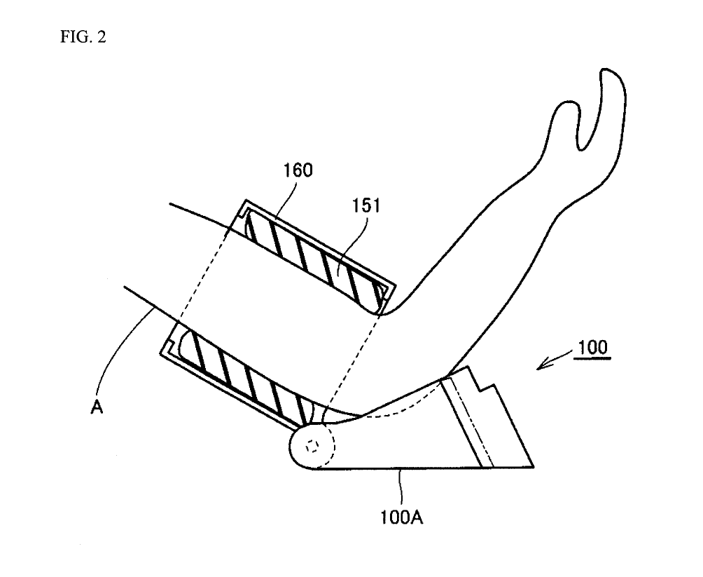

FIG. 2 is a schematic cross-sectional view of the sphygmomanometer 100 during blood pressure measurement. Referring to FIG. 2, when blood pressure is measured, the measurement subject inserts his upper arm inside the housing 160 and places his elbow on the aforementioned elbow rest, and indicates that measurement is to begin. Blood pressure is measured in the state in which the upper arm is fixed by compression by the aforementioned body compression fixing device. The body compression fixing device comprises a cuff 150. The cuff 150 encases an air bladder 151, which is the measuring fluid bag for compressing the measurement site and measuring blood pressure.

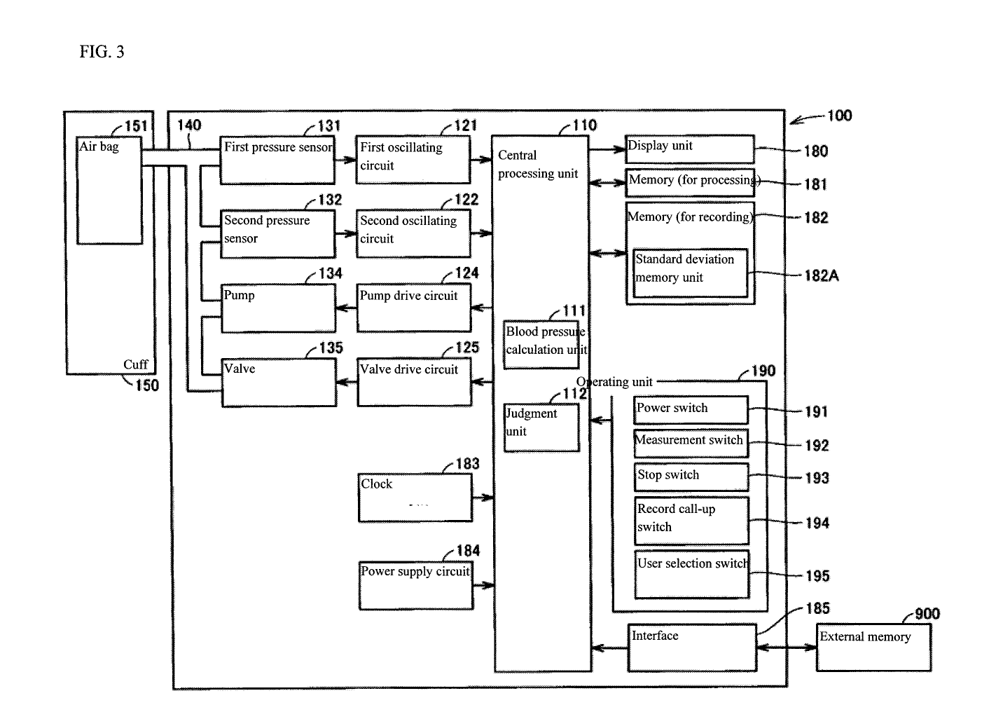

FIG. 3 is a block diagram of the sphygmomanometer 100.

Referring to FIG. 3, the sphygmomanometer 100 comprises a central processing unit 110 that centrally controls and monitors the elements in the sphygmomanometer 100. The central processing unit 110 comprises a CPU (central processing unit). As functions, the central processing unit 110 includes a blood pressure calculation unit 111 and a judgment unit 112. The blood pressure calculation unit 111 and judgment unit 112 may be constituted by the aforementioned CPU executing a prescribed program, or may be constituted as a circuit such as LSI (large scale integration).

Additionally, inside the main body 100A of the sphygmomanometer 100, an air system component for blood pressure measurement is provided, which supplies or exhausts air to or from the air bladder 151. The air system component for blood pressure measurement supplies air to the air bladder 151 and exhausts air from the air bladder 151 via an air tube 140. The air system component for blood pressure measurement comprises a first pressure sensor 131 and a second pressure sensor 132 for detecting the pressure in the air bladder 151, a pump 134 for inflating the air bladder 151, and a valve 135. In this embodiment, the first pressure sensor 131 and second pressure sensor 132 are capacitance pressure sensors, whose capacitance values vary depending on changes in the internal pressure of the air bladder 151. Inside the main body 100A are provided a first oscillating circuit 121 and second oscillating circuit 122 that generate oscillation frequency signals in accordance with the capacitance of the first pressure sensor 131 and second pressure sensor 132, a pump drive circuit 124 that drives the pump 134, and a valve drive circuit 125 that drives the valve 135.

Oscillation frequency signals are output from the first oscillating circuit 121 and second oscillating circuit 122 in accordance with changes in the internal pressure of the air bladder 151. By these signals being appropriately processed in the central processing unit 110 (blood pressure calculation unit 111), the blood pressure and pulse of the measurement subject are measured.

Furthermore, the sphygmomanometer 100 comprises memory 181 that is the work area of the central processing unit 110, memory 182 that stores programs that perform prescribed operations in the central processing unit 110 and various information such as measured blood pressure values, an operating unit 190 that is operated for entering various instructions for measurement and so forth, and a clock 183 that has a timing function.

The operating unit 190 comprises a power switch 191 that switches the power supply to the sphygmomanometer 100 on and off, a measurement switch 192 that is operated when blood pressure measurement is started in the sphygmomanometer 100, a stop switch 193 that is operated in order to stop a blood pressure measurement operation in progress, a record call-up switch 194 that is operated in order to display data stored in memory 182 such as blood pressure and pulse on the display unit 180, and a user selection switch 195 that selects the measurement subject by the sphygmomanometer 100.

The central processing unit 110 reads and writes information to a removable external memory 900 in the main body 100A, such as a floppy disk, USB (universal serial bus) memory or SD memory card. The sphygmomanometer 100 comprises an interface 185 for performing read/write processes from/to the external memory 900.

The sphygmomanometer 100 also comprises a power supply circuit 184 that supplies power to the elements in the sphygmomanometer 100.

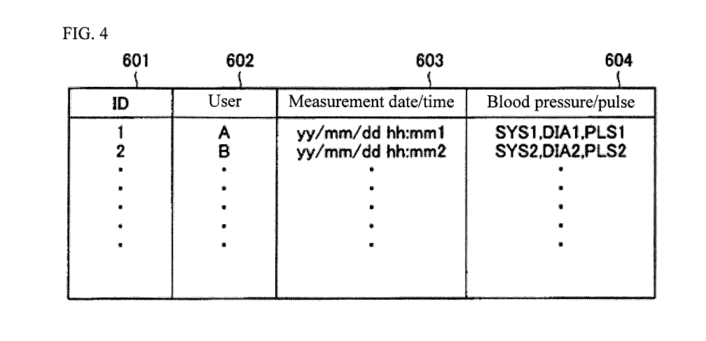

Memory 182 comprises a standard deviation memory unit 182A that stores the standard deviation values described below. Measurement record data related to the blood pressure and pulse of the user is also stored in memory 182. An example of this measurement data is shown in FIG. 4.

Referring to FIG. 4, in the measurement record data, an ID 601 that is information that specifies the set of measurement data, a user 602 that indicates the name of the user, a measurement date and time 603 that indicates the date and time at which the data was measured, and a blood pressure value/measurement value 604 that indicates the set of measurement data (maximum blood pressure value, minimum blood pressure value, pulse) are associated with each other. By referencing the measurement record data, the sphygmomanometer 100 can manage this history of measurement record data for each user.

The configuration of the first pressure sensor 131 will now be described. Note that the configuration of the second pressure sensor 132 can be the same as that of the first pressure sensor 131.

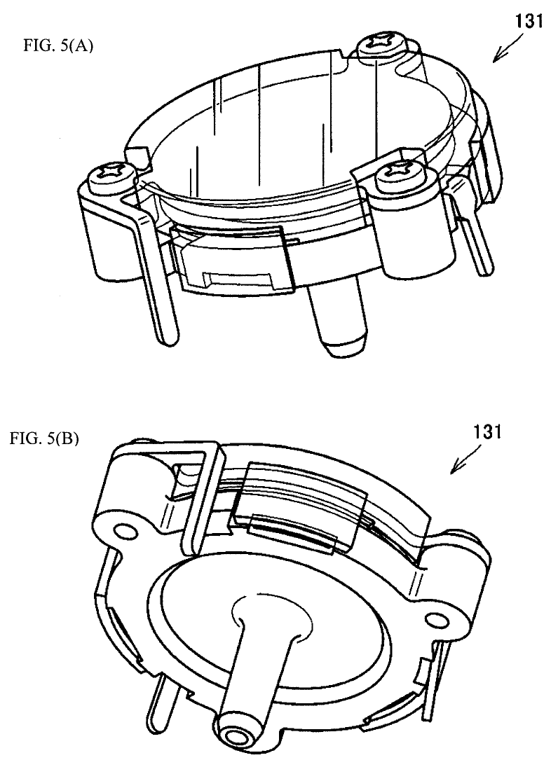

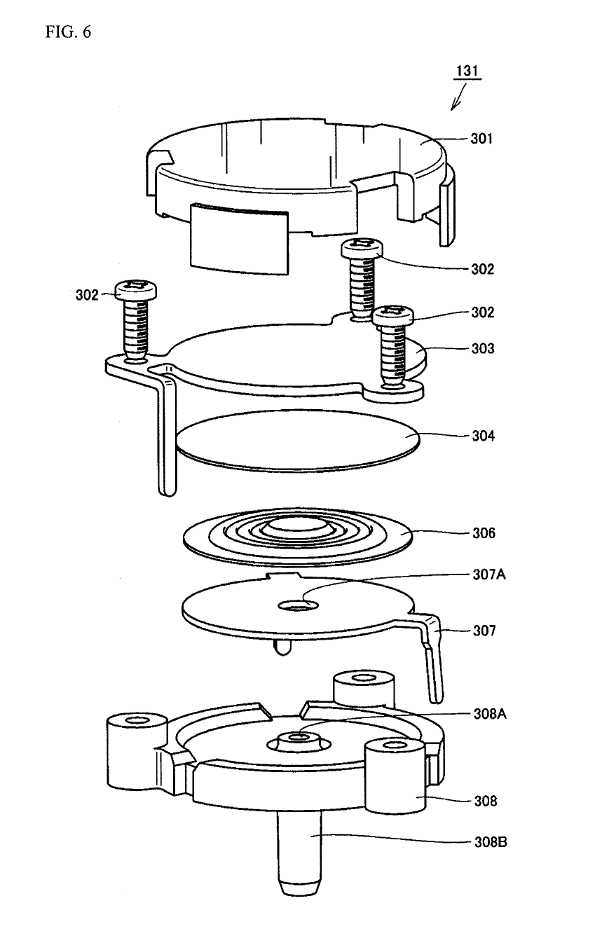

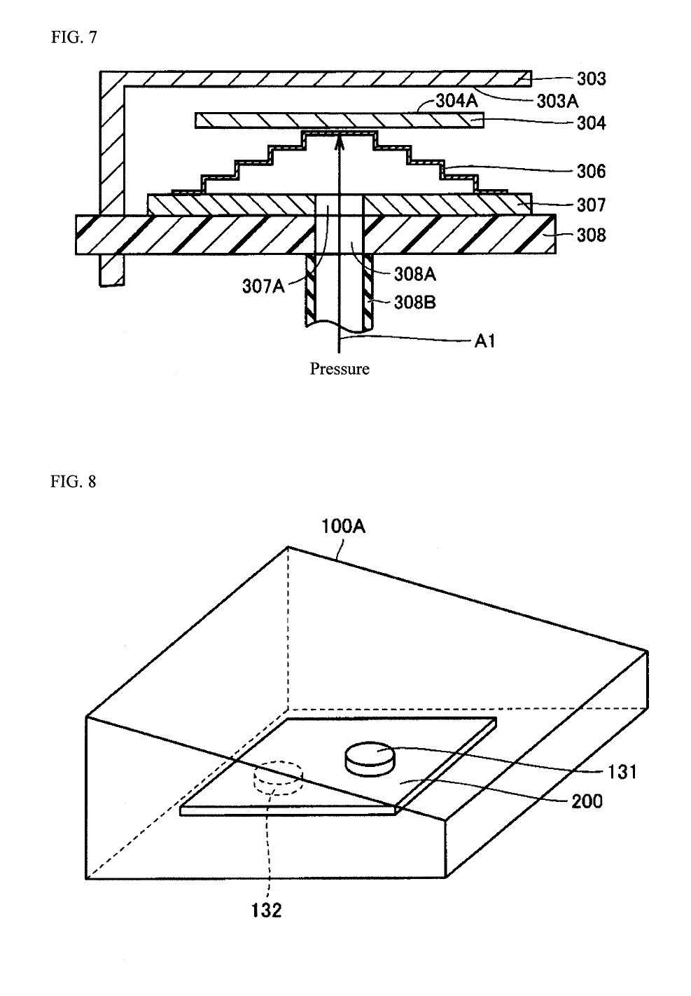

FIG. 5A and FIG. 5B show perspective views of the first pressure sensor 131. FIG. 6 shows an exploded perspective view of the first sensor 131. Additionally, FIG. 7 shows a schematic cross-sectional view of the first pressure sensor 131. In FIG. 7, illustrations of some of the parts shown in FIG. 6 are omitted.

Referring to FIG. 5A, FIG. 5B, FIG. 6 and FIG. 7, the first pressure sensor 131 comprises a first base 308, a second base 307 that is inlaid on the first base 308, a diaphragm 306 that is mounted on the second base 307, a moveable electrode 304 that is arranged on the diaphragm 306, and a fixed electrode 303 that is arranged on the moveable electrode 304. The diaphragm 306 and moveable electrode 304 are joined by soldering. The fixed electrode 303 is screwed into the first base 308 by screws 302 such that it is separated from the moveable electrode 304 at steady state (state in which pressure is not applied). A cap 301 is provided on the fixed electrode 303. The cap 301 is inlaid in the first base 308 from the top part such as the fixed electrode 303, such that it covers the outline of the first base 308 and the first pressure sensor 131.

A hole 308A is formed near the center of the first base 308. A tube 308B is formed on the bottom part of the first base 308. The hole 308A penetrates through to the bottom end of the tube 308B.

The tube 308B is connected to the air bladder 151 by an air tube 140. When a change in pressure occurs inside the air bladder 151, that change in pressure is transmitted to the first pressure sensor 131 via the air tube 140. In FIG. 7, the direction in which pressure is transmitted is indicated by arrow A1. Due to this change in pressure, the degree of expansion or contraction of the diaphragm 306 in the direction of arrow A1 changes.

The moveable electrode 304 and the fixed electrode 303 have faces 304A and 303A, respectively, which intersect with the direction of arrow A1. The face 304A is the face that is displaced by a change in pressure inside the air bladder 151.

The diaphragm 306 has a bellows structure, and its degree of expansion or compression in the direction of arrow A1 changes in accordance with the change in pressure inside the air bladder 151. The position of the face 304A in the direction of arrow A1 changes due to the aforementioned change in the degree of expansion or compression of the diaphragm 306. As a result, the distance of the face 304A from the face 303A in the direction of arrow A1 changes. As a result, the capacitance of the first pressure sensor 131 changes. The pressure inside the air bladder 151 is detected in the sphygmomanometer 100 based on the change in capacitance of the first pressure sensor 131. Note that information for converting the capacitance value of the first pressure sensor 131 to the pressure value inside the air bladder 151 is stored in memory 181.

The second pressure sensor 132 has the same configuration as the first pressure sensor, and it is connected to the air bladder 151 by the air tube 140. In the second pressure sensor 132 as well, similar to the first pressure sensor 131, the distance between the moveable electrode and fixed electrode changes in accordance with a change in pressure inside the air bladder 151, and as a result, the capacitance of the second pressure sensor 132 changes. The pressure inside the air bladder 151 can be detected in the sphygmomanometer 100 based on the change in capacitance of the second pressure sensor 132.

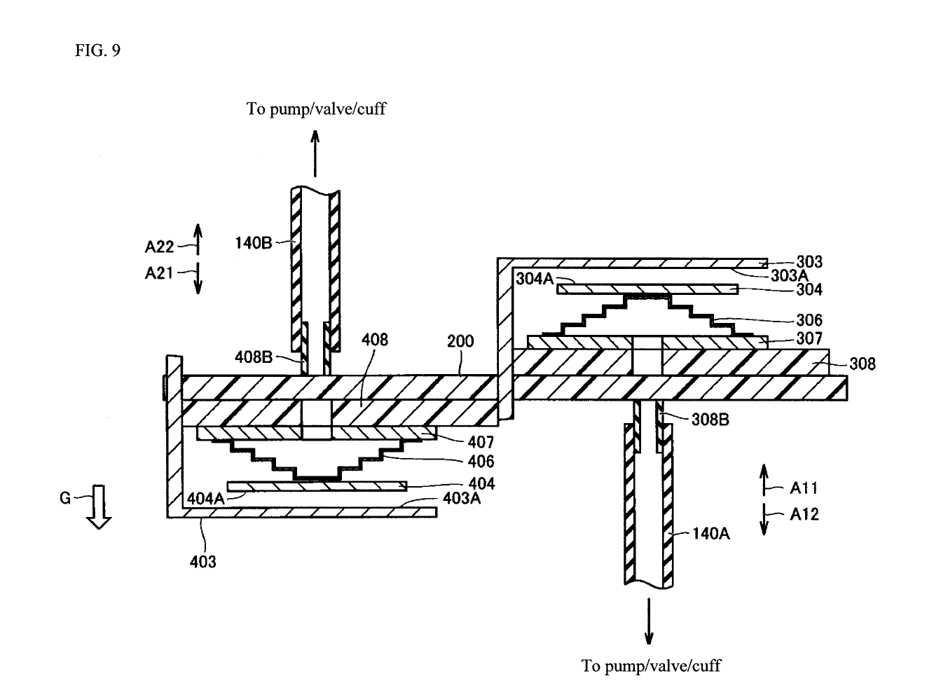

FIG. 8 and FIG. 9 are drawings for describing the arrangement of the first pressure sensor 131 and second pressure sensor 132 in the sphygmomanometer 100. The second pressure sensor 132 of FIG. 9, similar to the first pressure sensor 131, comprises a first base 408, a second base 407, a diaphragm 406, a moveable electrode 404, and a fixed electrode 403. The moveable electrode 404 has a face 404A. The fixed electrode 403 has a face 403A. The faces 404A and 403A are equivalent to the face 304A of the moveable electrode 304 and the face 303A of the fixed electrode 303, respectively. The first base 408, similar to the first base 308, has a hole 408A, which is equivalent to the hole 308A, and also has a tube 408B, which is equivalent to the tube 308B.

Referring to FIG. 8 and FIG. 9, a substrate 200 on which various parts are mounted is provided inside the main body 100A. In this embodiment, the first sensor 131 is mounted on the substrate 200 such that it penetrates the tube 308B from its top face, and the second sensor 132 is mounted such that it penetrates the tube 408 from its bottom face. The tube 308B is connected to an air tube 140A. The tube 408B is connected to an air tube 140B. The air tube 140A and air tube 140B both branch off from the air tube 140, and connect to the inside of the air bladder 151.

Arrows A11, A12, A21 and A22, respectively, indicate the directions in which the diaphragms 306 and 406 are displaced when the pressure changes inside the air bladder 151, and the directions in which the moveable electrodes 304 and 404 are displaced in accordance with displacement of the diaphragms 306 and 406. Specifically, when the pressure inside the air bladder 151 rises, the diaphragm 306 receives force in the direction of arrow A11 and is displaced in that direction. As a result, the face 304A is displaced in the direction of arrow A11 and moves closer to the face 303A. Additionally, when the pressure inside the air bladder 151 rises, the diaphragm 406 receives force in the direction of arrow A21 and is displaced in that direction. As a result, the face 404A is displaced in the direction of arrow A21 and moves closer to the face 403A.

On the other hand, when the pressure inside the air bladder 151 decreases, the diaphragm 306 receives force in the direction of arrow A12 and is displaced in that direction. As a result, the face 304A is displaced in the direction of arrow A12 and moves farther from the face 303A. Additionally, when the pressure inside the air bladder 151 decreases, the diaphragm 406 receives force in the direction of arrow A22 and is displaced in that direction. As a result, the face 404A is displaced in the direction of arrow A22 and moves farther from the face 403A.

Arrow G indicates the direction of gravity. Actually, the amount of displacement of the aforementioned diaphragms 306 and 406 and the amount of displacement of the faces 304A and 404A are affected by gravity in addition to the amount of change in pressure inside the air bladder 151.

If the pressure inside the air bladder 151 increases a certain fixed amount, the amount of displacement of the diaphragm 406 in the direction of arrow A21 and the amount of displacement of the face 404A in the direction of arrow A21 will be larger than the amount of displacement of the diaphragm 306 in the direction of arrow A11 and the amount of displacement of the face 304A in the direction of arrow A11. Arrow A21 points in the direction of the dead weight of the diaphragm 406 and the moveable electrode 404, whereas arrow A11 points in the direction opposite the dead weight of the diaphragm 306 and the moveable electrode 304.

If the pressure inside the air bladder 151 decreases a certain fixed amount, the amount of displacement of the diaphragm 306 in the direction of arrow A12 and the amount of displacement of the face 304A in the direction of arrow A12 will be larger than the amount of displacement of the diaphragm 406 in the direction of arrow A22 and the amount of displacement of the face 404A in the direction of arrow A22. Arrow A12 points in the direction of the dead weight of the diaphragm 406 and the moveable electrode 404, whereas arrow A22 points in the direction opposite the dead weight of the diaphragm 406 and the moveable electrode 404.

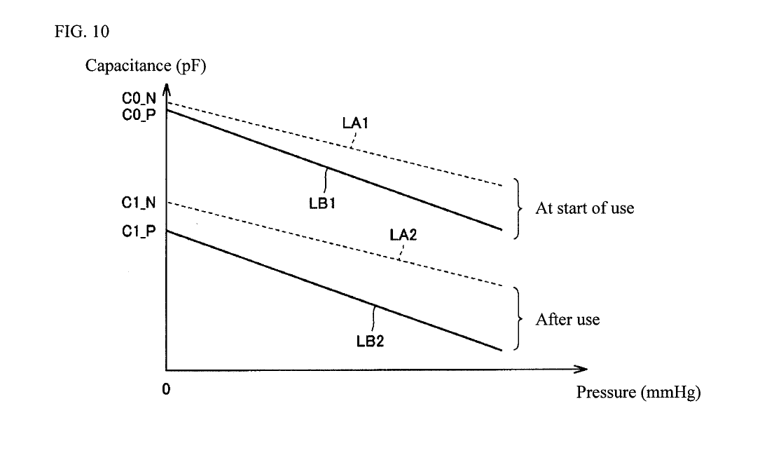

FIG. 10 is a graph showing an example of the changes in capacitance of the first pressure sensor 131 and second pressure sensor 132 when the pressure in the air bladder 151 changes. Note that in FIG. 10, line LA1 and line LA2 indicate the change in capacitance of the first pressure sensor 131, and line LB1 and line LB2 indicate the change in capacitance of the second pressure sensor 132. Also, line LA1 and line LB1 indicate the change in capacitance when use of the sphygmomanometer 100 begins, and line LA2 and line LB2 indicate the change in capacitance after the sphygmomanometer has been used for a prescribed time.

As is understood from FIG. 10, in the sphygmomanometer 100, when the internal pressure of the air bladder 151 rises, the capacitance of the first pressure sensor 131 and the capacitance of the second pressure sensor 132 decrease. The degree of decrease in capacitance in response to the rise in pressure inside the air bladder 151 is lower in the first pressure sensor 131 than in the second pressure sensor 132. This difference in the degrees of change is based on the difference in dead weight with respect to displacement of the diaphragms 306 and 406 and the moveable electrodes 304 and 404, due to the difference in the way the first pressure sensor 131 and second pressure sensor 132 are placed inside the main body 100A, as described in reference to FIG. 9.

Line LA2 and line LB2 indicate the changes in capacitance of the first sensor 131 and the second sensor 132 following the change in pressure inside the air bladder 151 at points in time after the start of use. Because the absolute value of capacitance changes depending on environmental factors such as ambient temperature, in this example the capacitance values since the start of use are all offset to a lower value. To compensate for the change in capacitance due to environmental factors, the offset amount is corrected when the sphygmomanometer is initialized.

Here, the capacitance of the first pressure sensor 131 and the capacitance of the second pressure sensor 132 are compared when the pressure inside the air bladder 151 is 0 mmHg. The capacitance values of the first pressure sensor 131 and second pressure sensor 132 with the pressure inside the air bladder 151 at 0 mmHg at the start of use are taken as C0_N and C0_P, respectively, the capacitance values of the first pressure sensor 131 and second pressure sensor 132 with the pressure inside the air bladder 151 at 0 mmHg at a point after time has passed since the start of use are taken as C1_N and C1_P, respectively, and the differences between them are taken as ΔC0 and ΔC1, respectively.

In this embodiment, when the difference in capacitance ΔC1 between the first pressure sensor 131 and the second pressure sensor 132 is compared with the difference ΔC0 at the start of use, if the difference between ΔC1 and ΔC0 exceeds a prescribed threshold value (called “TH” hereinafter), the sphygmomanometer 100 judges that an abnormal state has resulted from degradation of the diaphragms 306 and 406 and the moveable electrodes 304 and 404, and it reports that fact. Note that TH is equivalent to the aforementioned standard deviation, which is stored in the standard deviation memory unit 182A. Additionally, ΔC0 is also stored in memory 182 when shipped from the factory.

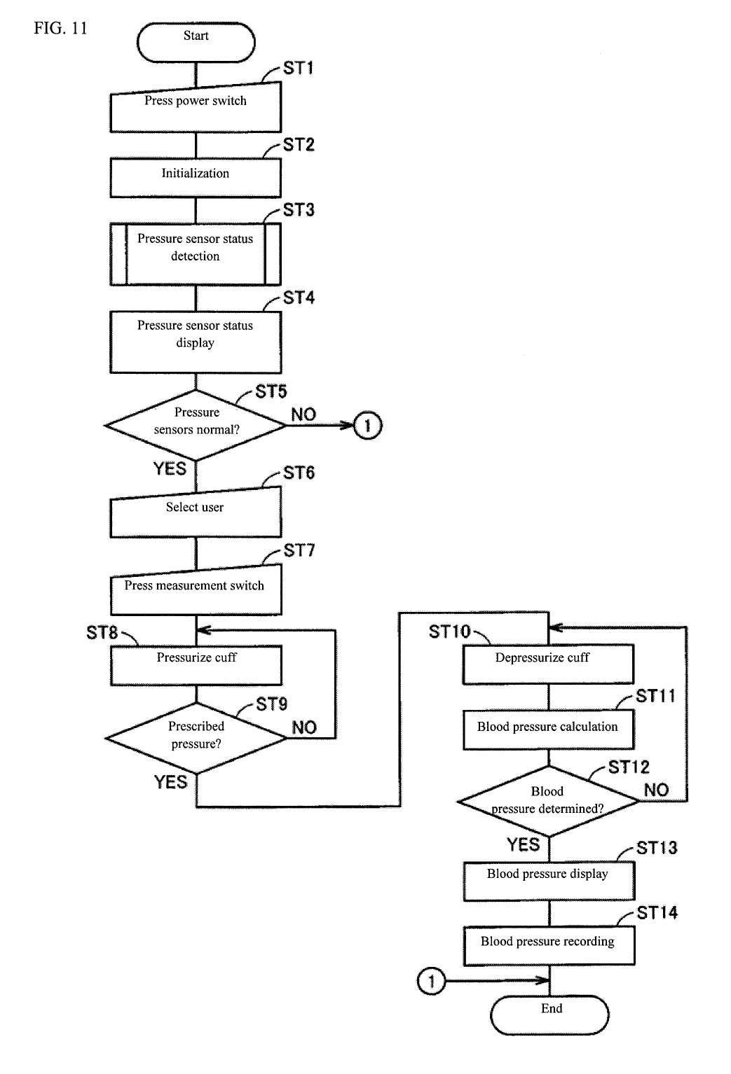

FIG. 11 is a flow chart of the process (blood pressure measurement process) executed when the blood pressure of the measurement subject is measured in the sphygmomanometer 100.

Referring to FIG. 11, when the power switch 191 is pressed (step ST1), the central processing unit 110 performs initialization of the sphygmomanometer 100 in step ST2, and then, in step ST3, it executes the pressure sensor status detection process.

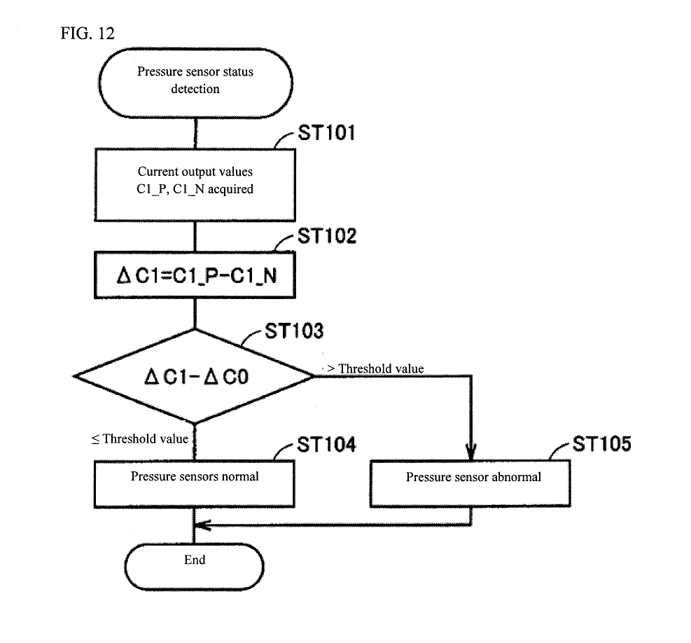

FIG. 12 is a flow chart of the pressure sensor status detection process subroutine. Referring to FIG. 12, in this process, first, in step ST101, the central processing unit 110 acquires the capacitance value of the first pressure sensor 131 (C1_N) and the capacitance value of the second pressure sensor 132 (C1_P) at that time, and advances the process to step ST102.

In step ST102, the central processing unit 110 calculates AC1, which is the difference between the aforementioned C1_N and C1_P, based on the following formula.

ΔC1=C1_{—}P−C1_{—}N (1)

Then, in step ST103, the central processing unit 110 calculates the difference between ΔC1 calculated in step ST102 and ΔC0 stored in memory 182, and if this difference is less than or equal to the threshold value (standard deviation TH stored in standard deviation memory unit 182A), it advances the process to step ST104; if the difference exceeds the threshold value, it advances the process to step ST105.

In step ST104, the central processing unit 110 considers the first pressure sensor 131 and the second pressure sensor 132 to be normal, and returns the process to FIG. 11.

On the other hand, in step ST105, the central processing unit 110 considers at least one of the first pressure sensor 131 and second pressure sensor 132 to be abnormal, and returns the process to FIG. 11.

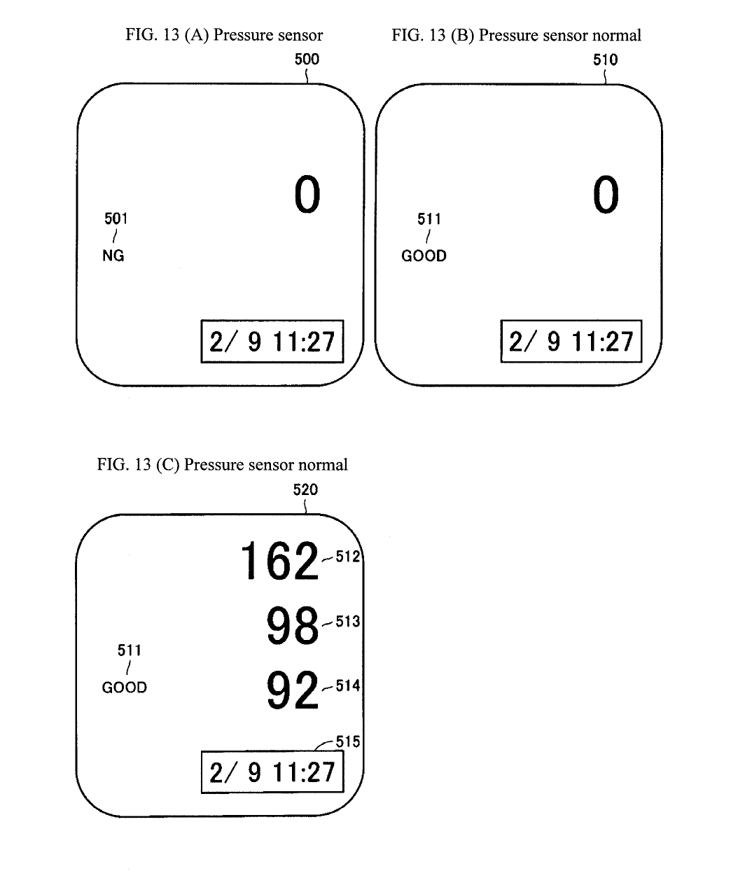

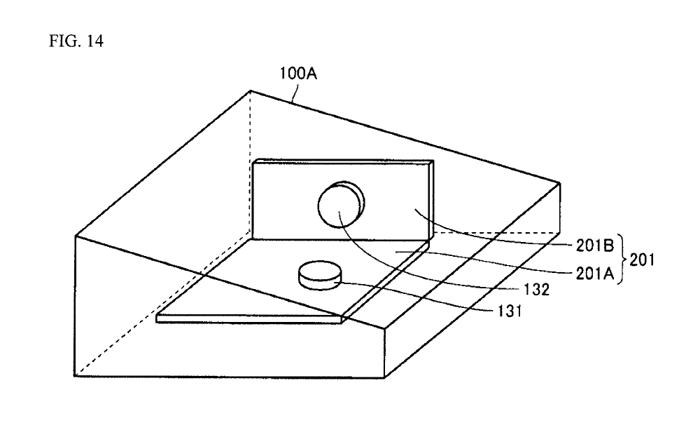

Returning to FIG. 11, after executing the pressure sensor status detection process in step ST3, in step ST4, the central processing unit 110 displays the status of the first pressure sensor 131 and the second pressure sensor 132 detected in step ST3 on the display unit 180, and advances the process to step ST5. FIG. 13A and FIG. 13B show sample displays of the display unit 180 in step ST4.

FIG. 13A shows a display screen 500 when a sensor was judged to be abnormal in step ST105, and FIG. 13B shows a display screen 510 when the sensors were judged to be normal in step ST104.

The display screen 510 of FIG. 13B displays a character string 511 which indicates the status (GOOD) of the first pressure sensor 131 and second pressure sensor 132, the current pressure inside the air bladder 151, and the current date and time 515.

The display screen 500 of FIG. 13A displays the current date and time and the current pressure in the air bladder, as well as a character string 501 which indicates the status (NG) of the first pressure sensor 131 and second pressure sensor 132.

Returning to FIG. 11, in step ST5, the central processing unit 110 determines the process to be executed next depending on whether the status of the first pressure sensor 131 and the second pressure sensor 132 judged in the pressure sensor status detection process in step ST3 was normal or abnormal. If the status was normal, it advances the process to step ST6, and if abnormal, it ends the blood pressure measurement process, leaving the display of step ST4 as is. As a result, the sphygmomanometer 100 displays an abnormality of the first pressure sensor 131 and second pressure sensor 132 on the display unit 180, and does not perform measurement of blood pressure and so forth.

In step ST6, the central processing unit 110 accepts operations regarding the measurement subject via the user selection switch. An example of such as operation is the entry of information that specifies to the sphygmomanometer 100 the measurement subject whose blood pressure and so forth are to be measured afterward.

In step ST7, the central processing unit 100 accepts operations regarding the measurement subject via the measurement switch 192.

Then, in step ST8, the central processing unit 110 pressurizes the cuff (air bladder 151). This pressurization is continued until the pressure inside the air bladder 151 in step ST9 reaches a prescribed pressure, and when it judges that the prescribed pressure has been reached, it advances the process to step ST10.

In step ST10, it starts depressurizing the cuff (air bladder 151), and advances the process to step ST11.

In step ST11, the central processing unit 110 calculates the blood pressure of the measurement subject based on the pressure value of the first pressure sensor 131 and/or the second pressure sensor 132 while being depressurized. It continues this blood pressure calculation until it judges that the blood pressure of the measurement subject has been determined in step ST12, and when it judges that the blood pressure value has been determined, it advances the process to step ST13.

In step ST13, the blood pressure value determined in step ST12 is displayed on the display unit 180 as shown on display screen 510 of FIG. 13C, and it advances the process to step ST14. Note that on display screen 520 of FIG. 13C, the maximum blood pressure 512, the minimum blood pressure 513 and the pulse 514 of the current measurement subject, and the current date and time 515 are shown. Here, in step ST12 described below, up to the time when updated measurement values have been determined, the maximum blood pressure 512, minimum blood pressure 513 and pulse 514 may not necessarily be displayed, or the previous measurement values of the current measurement subject may be displayed. Note that if the previous measurement values are displayed, it is preferred that there is also a display that warns that the displayed measurement values are not the current measurements but are the previous measurements.

In step ST14, the blood pressure value determined in step ST12 is stored in memory 182 in association with the measurement subject and the date and time of measurement, and the blood pressure measurement process is ended.

In the embodiment described above, a plurality of detection elements (first pressure sensor 131 and second pressure sensor 132) are placed in configurations such that gravity affects their detection output in mutually different ways. Thus, it can be judged whether or not at least one of the aforementioned plurality of detection elements is abnormal, based on whether or not it is within the assumed range (standard deviation TH) due to the difference in the effect of gravity.

### Second Embodiment

In the embodiment described above, a plurality of pressure sensors (first pressure sensor 131 and second pressure sensor 132) are placed in a configuration in which the faces that receive the pressure changes in the air bladder 151 differ from each other, as described primarily in reference to FIG. 9. In these pressure sensors, the capacitance changes in accordance with the pressure inside the air bladder 151, as described primarily in reference to FIG. 10. Thus, the difference between the difference in capacitance after a change (ΔC1) and the difference at the start of use (ΔC0) is calculated (ΔC1-ΔC0), and if this difference exceeds a threshold, at least one of the plurality of pressure sensors is considered abnormal, and this fact is reported and blood pressure is not measured.

The pressure sensor status detection process described in reference to FIG. 12 is a process that judges whether or not at least one of the plurality of pressure sensors (detection elements) is abnormal. In short, this process is executed by the judgment unit 112.

Note that when judging whether a pressure sensor is abnormal, instead of calculating the difference between ΔC0 and ΔC1, it is acceptable to pre-measure the capacitance of the first pressure sensor 131 at the start of use (C0_N) and the capacitance of the second pressure sensor 132 at the start of use (C0_P), store these in memory, and then compare these with the capacitance values of the sensors during measurement. Specifically, it is acceptable to acquire the capacitance of the first pressure sensor 131 during measurement (C1_N) and the capacitance of the second pressure sensor 132 during measurement (C1_P), and to judge them to be abnormal if the difference between C0_N and C1_N exceeds a first threshold value or the difference between C0_P and C1_P exceeds a second threshold value. Note that if the difference between C0_N and C1_N exceeds the first threshold value but the difference between C0_P and C1_P does not exceed the second threshold value, it is acceptable to judge that only the first pressure sensor 131 is abnormal, and to perform blood pressure measurement based on the detection output of the second pressure sensor 132. Also, if the difference between C0_P and C1_P exceeds the second threshold value but the difference between C0_N and C1_N does not exceed the first threshold value, it is acceptable to judge that only the second pressure sensor 132 is abnormal, and to perform blood pressure measurement based on the detection output of the first pressure sensor 131.

### Third Embodiment

In the embodiments described above, a plurality of pressure sensors (first pressure sensor 131 and second pressure sensor 132) are attached such that the directions of displacement of the faces inside the pressure sensor in accordance with changes in pressure inside the air bladder 151 differ by 180 degrees (for example, arrow A11 and arrow A21), as described primarily in reference to FIG. 9. Note that the attachment configuration of the plurality of pressure sensors in the blood pressure measurement device according to one or more embodiments of the present invention is not limited thereto. The plurality of pressure sensors may be attached such that they differ by 90 degrees, as shown in FIG. 14, provided that they are attached such that the directions of displacement of their faces in accordance with changes in pressure in the air bladder 151 differ from each other.

Specifically, in the main body 100A shown in FIG. 14, the substrate 201 has a first face 201 A and a second face 201 B provided at an angle of 90 degrees with respect to the first face 201A. The first pressure sensor 131 is mounted on the first face 201A, and the second pressure sensor 132 is mounted on the second face 201B.

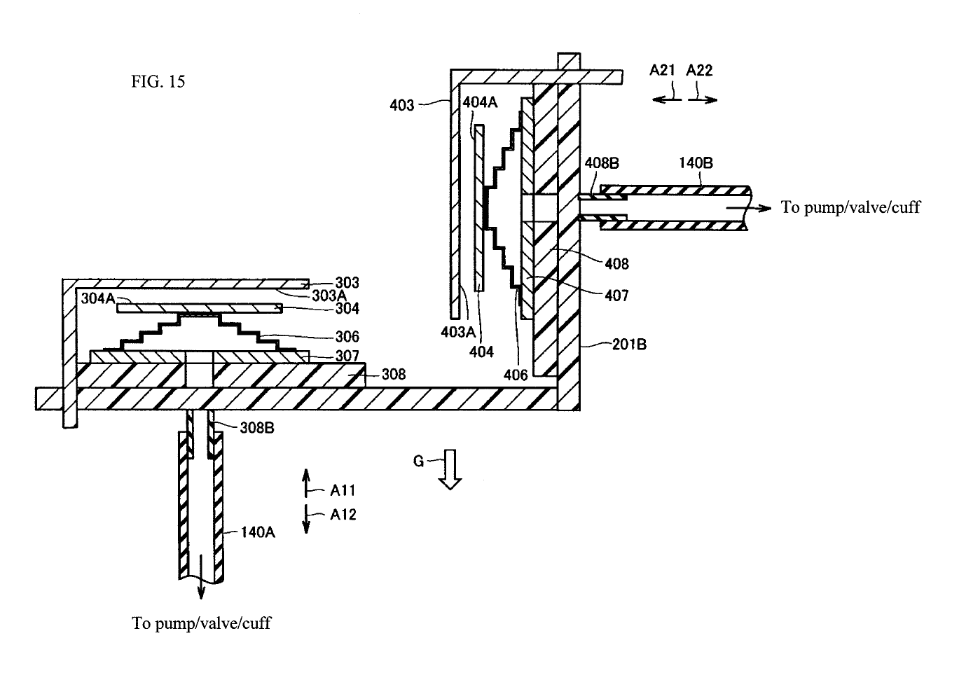

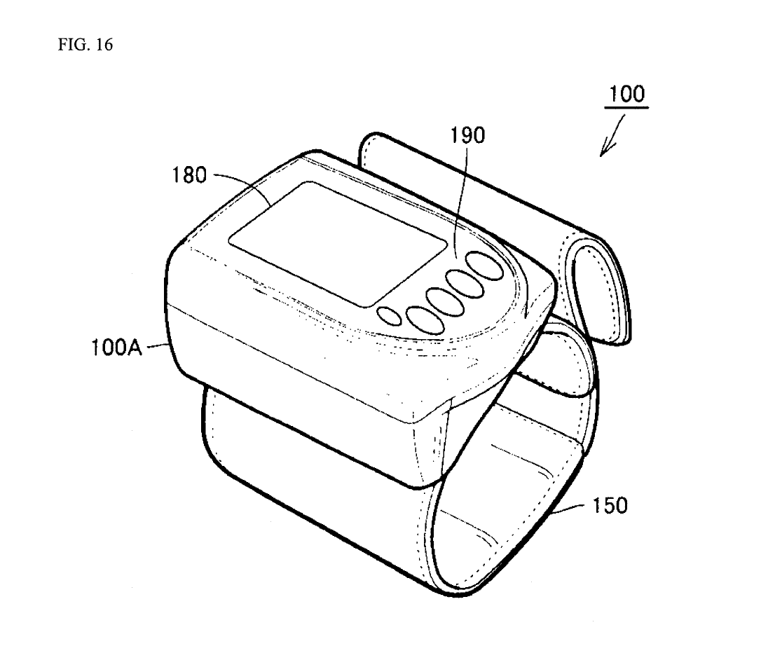

FIG. 15 shows a schematic cross-sectional view of the first pressure sensor 131 and the second pressure sensor 132 of this embodiment.

Referring to FIG. 15, as described in reference to FIG. 9, when the pressure inside the air bladder 151 rises, the diaphragm 306 is displaced in the direction of arrow A11. As a result, the face 304A is displaced in the direction of arrow A11 and moves closer to the face 303A. Additionally, in this case, the diaphragm 406 is displaced in the direction of arrow A21. As a result, the face 404A is displaced in the direction of arrow A21 and moves closer to the face 403A.

When the pressure inside the air bladder 151 decreases, the diaphragm 306 is displaced in the direction of arrow A12. As a result, the face 304A is displaced in the direction of arrow A12 and moves farther from the face 303A. Additionally, in this case, the diaphragm 406 is displaced in the direction of arrow A22. As a result, the face 404A is displaced in the direction of arrow A22 and moves farther from the face 403A.

Arrow G indicates the direction of gravity, similar to FIG. 9.

Arrow A11 and arrow A21 form an angle of 90 degrees. Arrow A12 and A22 also form an angle of 90 degrees. Thus, the way that the dead weight of the diaphragm 306 contributes to displacement of the diaphragm 306 in the direction of arrow A11 and the direction of arrow A12 differs from the way that the dead weight of the diaphragm 406 contributes to displacement of the diaphragm 406 in the direction of arrow A21 and the direction of arrow A22. Also, the way that the dead weight of the moveable electrode 304 contributes to displacement of the moveable electrode 304 in the direction of arrow A11 and the direction of arrow A12 differs from the way that the dead weight of the moveable electrode 404 contributes to displacement of the moveable electrode 404 in the direction of arrow A21 and the direction of arrow A22.

Therefore, in this embodiment as well, because it is assumed that a difference arises between the change in capacitance of the first pressure sensor 131 and the second pressure sensor 132 in response to a fixed change in pressure in the air bladder 151, the presence or absence of an abnormality in the plurality of pressure sensors can be detected based on this difference.

Note that in both FIG. 9 and FIG. 15, in at least one of the pressure sensors, the displacement direction of the face runs along the direction of gravity, but the attachment configuration is not limited to this. Any of the plurality of pressure sensors may be attached so as not to run along the direction of gravity.

### Fourth Embodiment

In the embodiments described above, the sphygmomanometer 100 is of the type in which the main body 100A is mounted on a table or the like and the measurement subject inserts the site to be measured (arm), but the blood pressure measurement device according one or more embodiments of the present invention is not limited to this type.

For example, the main body and the cuff of the sphygmomanometer 100 may be integrally constructed, as shown in FIG. 16.

### Fifth Embodiment

In the embodiments described above, the pressure sensors are capacitance pressure sensors, but the blood pressure measurement device according to one or more embodiments of the present invention is not limited thereto.

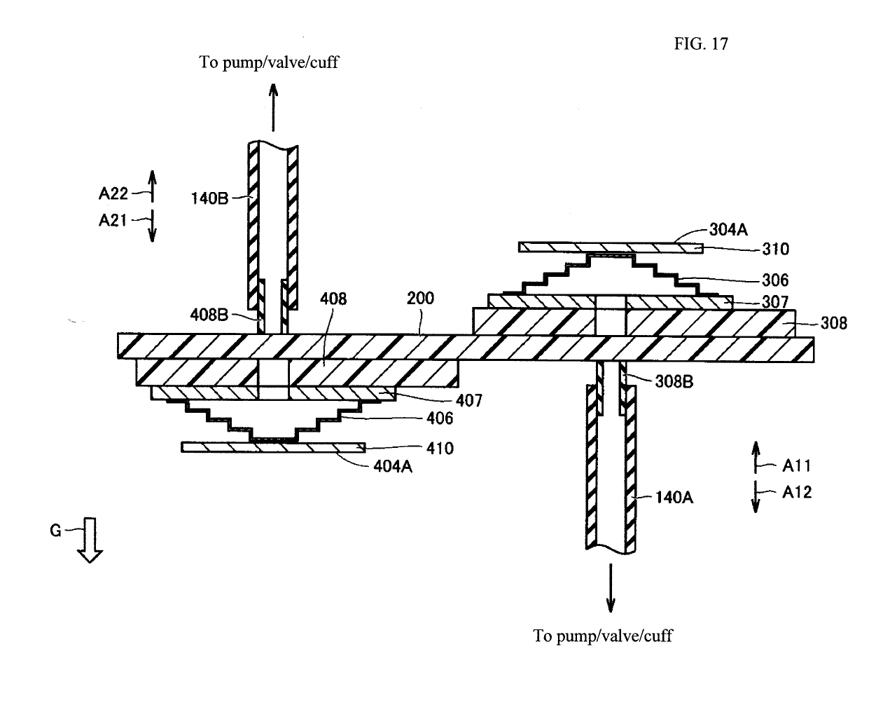

For example, instead of the moveable electrode 304 and fixed electrode 303 in the first pressure sensor 131, a piezoelectric element 310 may be provided as shown in FIG. 17. Also, instead of the moveable electrode 404 and fixed electrode 403 in the second pressure sensor 132, a piezoelectric element 410 may be provided.

In the first pressure sensor 131 of the sphygmomanometer of this embodiment, pressure changes inside the air bladder 151 are transmitted to the diaphragm 306 via the air tube 140A. As a result, the degree of expansion or contraction of the diaphragm 306 changes, and the piezoelectric element 310 is deformed. In the first pressure sensor 131, the change in resistance of the piezoelectric element 310, which changes accompanying deformation of the piezoelectric element 310, is detected (by a circuit not shown in diagram), and the change in pressure inside the air bladder 151 is thereby detected.

In the second pressure sensor 132, pressure changes inside the air bladder 151 are transmitted to the diaphragm 406 via the air tube 140B. As a result, the degree of expansion or contraction of the diaphragm 406 changes, and the piezoelectric element 410 is deformed. In the second pressure sensor 132, the change in resistance of the piezoelectric element 410, which changes accompanying deformation of the piezoelectric element 410, is detected (by a circuit not shown in diagram), and the change in pressure inside the air bladder 151 is thereby detected.

When the pressure inside the air bladder 151 rises, the diaphragm 306 and the piezoelectric element 310 are displaced in the direction of arrow A11, and the diaphragm 406 and piezoelectric element 410 are displaced in the direction of arrow A21. Displacement in the direction of arrow A11 is displacement in the direction opposite gravity, whereas displacement in the direction of arrow A21 is displacement that runs along the direction of gravity. Thus, it is thought that even when the pressure inside the air bladder 151 rises a fixed amount, the amounts of displacement of the piezoelectric element 310 and piezoelectric element 410 differ because the effects of dead weight on the piezoelectric element 310 and the piezoelectric element 410 differ.

In this embodiment, the presence of an abnormality in a plurality of pressure sensors is detected based on a difference in the amounts of displacement as described above.

### Sixth Embodiment

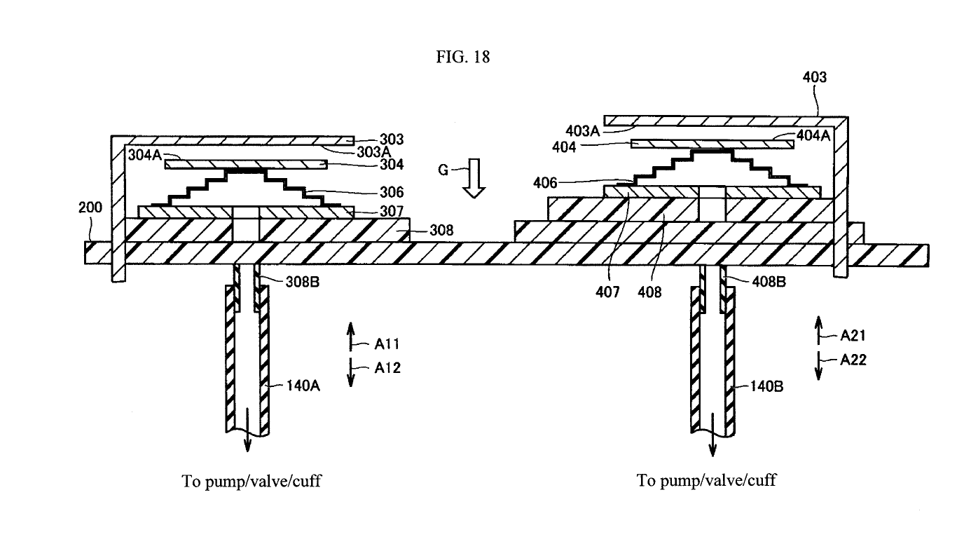

In this embodiment, as illustrated in FIG. 18, in the first pressure sensor 131 and second pressure sensor 132, the faces that are displaced accompanying a change in pressure in the air bladder 151 (face 304A and face 404A) have the same displacement direction, but they are arranged such that their heights are different. The magnitude of gravity applied differs in accordance with height. Thus, even with this arrangement, even when the same change in pressure occurs in the air bladder 151, the amount of displacement of the diaphragm 306 and the amount of displacement of the diaphragm 406 differ in the pressure sensor 131 and pressure sensor 132, and also, the amount of displacement of the moveable electrode 304 and the amount of displacement of the moveable electrode 404 differ.

In this embodiment, the presence of an abnormality in a plurality of pressure sensors is detected based on a difference in the amounts of displacement as described above.

### Advantage of the Invention

Because a plurality of pressure sensors are arranged such that weight of the sensors onto the diaphragms of respective pressure sensors become different from each other, displacement of the diaphragms in response to the change of internal air pressure of the air bladder would be different from each other. As a result, output of these pressure sensors would be different from each other. That difference can be used to determine whether the sensor is working normally without failure. Because of the different condition/output of the plurality of pressure sensors, even if all of the sensors failed, the resulting output from those sensors are less likely to become the same. Therefore, the device can detect the failure of the pressure sensor more reliably than the blood pressure measurement device having pressure sensors that are arranged in a same condition or state.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A blood pressure measurement device comprising:
a cuff containing an air bladder for wrapping around a measurement area of a subject;
an air charger that inflates the air bladder;
an air discharger that deflates the air bladder;
a sensor that detects a change of an internal pressure of the air bladder during the inflation and/or deflation of the air bladder, the sensor further comprising:
a first sensor comprising a diaphragm connected to the air bladder,
wherein a face of the diaphragm is flexibly displaced in accordance with the change of the internal pressure of the air bladder; and
a second sensor comprising a diaphragm connected to the air bladder,
wherein a face of the diaphragm is flexibly displaced in accordance with the change of the internal pressure of the air bladder,
wherein the face of the diaphragm of the second sensor is arranged in a different position and/or direction from the face of the diaphragm of the first sensor; and
a main body comprising a central processing unit that calculates the blood pressure of a measurement subject from the change of the internal pressure of the air bladder detected by the sensor, the central processing unit further comprising a failure judgment unit that determines whether there was any failure with the sensor,
wherein upon receiving the internal pressure of the air bladder detected by the first sensor and the second sensor, the failure judgment unit determines whether a difference between the internal pressures detected by the first sensor and the second sensor is within a predetermined range, and if the difference is beyond the predetermined range, the failure judgment unit determines that the sensor failed to perform normal detection of the internal pressure of the air bladder.

2. The blood pressure measurement device according to claim 1, wherein upon receiving the change of internal pressure of the air bladder, the face of diaphragm of the first sensor is displaced in a different direction from the face of diaphragm of the second sensor.

3. The blood pressure measurement device according to claim 2, wherein the face of diaphragm of the first sensor is displaced in an opposite direction from the face of diaphragm of the second sensor.

4. The blood pressure measurement device according to claim 2, wherein the face of diaphragm of the first sensor is displaced in a perpendicular direction from the face of diaphragm of the second sensor.

5. The blood pressure measurement device according to claim 1, wherein upon receiving the change of internal pressure of the air bladder, diaphragm of the first sensor is displaced by a different height from the diaphragm of the second sensor.

6. The blood pressure measurement device according to claim 1, wherein the first sensor and the second sensor are both capacitance pressure sensors, and capacitance of the first and second sensors are changed by the displacement of the diaphragms in accordance with the change of the internal pressure of the air bladder.

7. The blood pressure measurement device according to claim 1, wherein the first and second pressure sensors are both semiconductor pressure sensors that comprise a piezoelectric element attached to the diaphragms, and resistance of the first and second sensors are changed by the displacement of the diaphragms in accordance with the change of the internal pressure of the air bladder.

8. A blood pressure measurement device comprising:
means for wrapping an air bladder around an measurement area of a subject;
means for inflating the air bladder;
means for deflating the air bladder;
means for detecting a change of an internal pressure of the air bladder during the inflation and/or deflation of the air bladder, the means further comprising:
a first sensor comprising a diaphragm connected to the air bladder,
wherein a face of the diaphragm is flexibly displaced in accordance with the changes of the internal pressure of the air bladder; and
a second sensor comprising a diaphragm connected to the air bladder,
wherein a face of the diaphragm is flexibly displaced in accordance with the change of the internal pressure of the air bladder,
wherein the face of the diaphragm of the second sensor is arranged in a different position and/or direction from the face of the diaphragm of the first sensor; and
means for calculating the blood pressure of a measurement subject from the change of the internal pressure of the air bladder detected by the detecting means, the calculating means further comprising means for determining whether there was any failure with the sensor,
wherein upon receiving the internal pressure of the air bladder detected by the first sensor and the second sensor, it is determined whether a difference between the internal pressures detected by the first sensor and the second sensor is within a predetermined range, and if the difference is beyond the predetermined range, the sensor is found to have failed to perform normal detection of the internal pressure of the air bladder.

9. The blood pressure measurement device according to claim 8, wherein upon receiving the change of internal pressure of the air bladder, the face of diaphragm of the first sensor is displaced in a different direction from the face of diaphragm of the second sensor.

10. A method of detecting a failure of a sensor of a blood pressure measurement device, the method comprising:
wrapping an air bladder around a measurement area of a subject;
inflating and/or deflating the air bladder;
detecting a change of an internal pressure of the air bladder during the inflation and/or deflation of the air bladder by using a sensor, the sensor comprising:
a first sensor comprising a diaphragm connected to the air bladder,
wherein a face of the diaphragm is flexibly displaced in accordance with the change of the internal pressure of the air bladder; and
a second sensor comprising a diaphragm connected to the air bladder,
wherein a face of the diaphragm is flexibly displaced in accordance with the change of the internal pressure of the air bladder,
wherein the face of the diaphragm of the second sensor is arranged in a different position and/or direction from the face of the diaphragm of the first sensor;
determining whether a difference between the internal pressures detected by the first sensor and the second sensor is within a predetermined range; and
if the difference is beyond the predetermined range, determining that the sensor has failed to perform normal detection of the internal pressure of the air bladder.

11. The method according to claim 10, wherein upon receiving the change of internal pressure of the air bladder, the face of diaphragm of the first sensor is displaced in a different direction from the face of diaphragm of the second sensor.
